(19) United States
(12) Patent Application Publication
(10) Pub. No.: US 2026/0228617 A1
(43) Pub. Date: Aug. 6, 2026

(54) MACHINE LEARNING DEVICE, MACHINE LEARNING METHOD, PROGRAM, AND MACHINE LEARNING SYSTEM

(71) Applicant: NEC CORPORATION, Tokyo (JP)

(72) Inventors: Kiichi OBUCHI, Ibaraki (JP); Fumihiko KOSAKA, Ibaraki (JP)

(73) Assignee: NEC CORPORATION, Tokyo (JP)

(21) Appl. No.: 19/152,979

(22) PCT Filed: Jan. 24, 2024
(86) PCT No.: PCT/JP2024/002066

§ 371 Date: Aug. 1, 2025

(30) Foreign Application Priority Data
Feb. 9, 2023 (JP) 2023-018739

(51) Int. Cl. G06N 20/00; G16C 20/10; G16C 20/70

(52) U.S. Cl. CPC G06N 20/00; G16C 20/10; G16C 20/70

(57) ABSTRACT

The purpose of the present invention is to provide a machine learning device, a machine learning method, a program, and a machine learning system with which it is possible to predict high performance catalysts. A machine learning device according to the present disclosure is designed to predict catalyst performance, said device comprising: an acquisition unit that acquires a data set of interest including intended catalytic reaction information and a plurality of data sets including catalytic reaction information that is different from the intended catalytic reaction; a determination unit that determines commonality regarding catalytic reactions between the data set of interest and each of the plurality of data sets; and a training unit that trains a prediction model that predicts catalyst performance, using a data set selected from the plurality of data sets on the basis of the determination result and the data set of interest.

### TECHNICAL FIELD

The present invention relates to a machine learning device, a machine learning method, a program, and a machine learning system, and more particularly, to a machine learning device, a machine learning method, a program, and a machine learning system in searching for a catalyst composition.

### BACKGROUND ART

PTL 1 discloses a machine learning method using a constructed mathematical model for prediction of activity that is an objective characteristic.

### CITATION LIST

### Patent Literature

PTL 1: JP 2022-064628 A

### SUMMARY OF INVENTION

As disclosed in PTL 1, data collection and use of artificial intelligence (AI) have been advanced for prediction of activity that is an objective characteristic. On the other hand, prediction of search for a catalyst composition requires a sufficient number of samples in the same catalytic reaction, but new development has a problem that there is a little catalyst data.

In machine learning with less data, it is effective to perform transfer learning using similar data, but it is very difficult to extract similar data from data of many chemical reactions.

The present disclosure has been made in view of this, and an object thereof is to provide a machine learning device, a machine learning method, and a program capable of predicting a high-performance catalyst.

A machine learning device according to the present disclosure is designed to predict catalyst performance, and includes an acquisition unit that acquires a target data set including information on an intended catalytic reaction and a plurality of data sets including information on catalytic reactions different from the intended catalytic reaction, a determination unit that determines commonality regarding catalytic reactions between the target data set and each of the plurality of data sets, and a learning unit that trains a prediction model for predicting catalyst performance from the plurality of data sets by using a data set selected based on the determination result and the target data set.

A machine learning method according to the present disclosure is designed to predict catalyst performance, and includes acquiring a target data set including information on an intended catalytic reaction and a plurality of data sets including information on catalytic reactions different from the intended catalytic reaction, determining commonality regarding catalytic reactions between the target data set and each of the plurality of data sets, and training a prediction model for predicting catalyst performance from the plurality of data sets by using a data set selected based on the determination result and the target data set.

A program according to the present disclosure is a program for a machine learning device that predicts catalyst performance, the program causing a computer included in a machine learning device to execute, a step of acquiring a target data set including information on an intended catalytic reaction and a plurality of data sets including information on catalytic reactions different from the intended catalytic reaction, a step of determining commonality regarding catalytic reactions between the target data set and each of the plurality of data sets, and a step of training a prediction model for predicting catalyst performance from the plurality of data sets by using a data set selected based on the determination result and the target data set.

A machine learning system according to the present disclosure is designed to predict catalyst performance, and includes a server and a machine learning device, wherein the machine learning device includes an acquisition unit that acquires a target data set including information on an intended catalytic reaction and a plurality of data sets including information on catalytic reactions different from the intended catalytic reaction from the server, a determination unit that determines commonality regarding catalytic reactions between the target data set and each of the plurality of data sets, and a learning unit that trains a prediction model for predicting catalyst performance from the plurality of data sets by using a data set selected based on the determination result and the target data set.

According to the present disclosure, it is possible to provide a machine learning device, a machine learning method, and a program capable of predicting a high-performance catalyst.

### BRIEF DESCRIPTION OF DRAWINGS

FIG. 1 is a configuration diagram of a machine learning device according to the present disclosure.

FIG. 2 is a flowchart showing processing of the machine learning device according to the present disclosure.

FIG. 3 is a diagram illustrating a data set of an input unit according to the present disclosure.

FIG. 4 is a diagram illustrating a data set of the input unit according to the present disclosure.

FIG. 5 is a diagram illustrating usage rate ranking according to the present disclosure.

FIG. 6 is a diagram illustrating statistics for a data set according to the present disclosure.

FIG. 7 is a configuration diagram of a machine learning device according to the present disclosure.

### EXAMPLE EMBODIMENT

### Example Embodiment 1

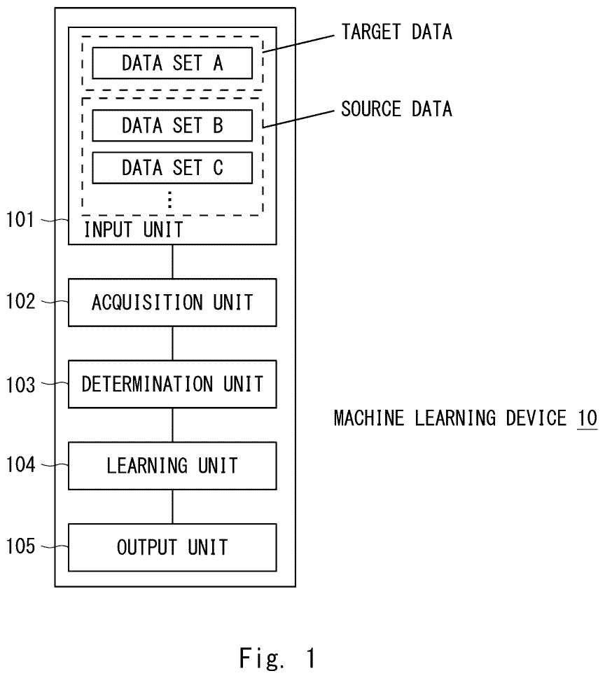

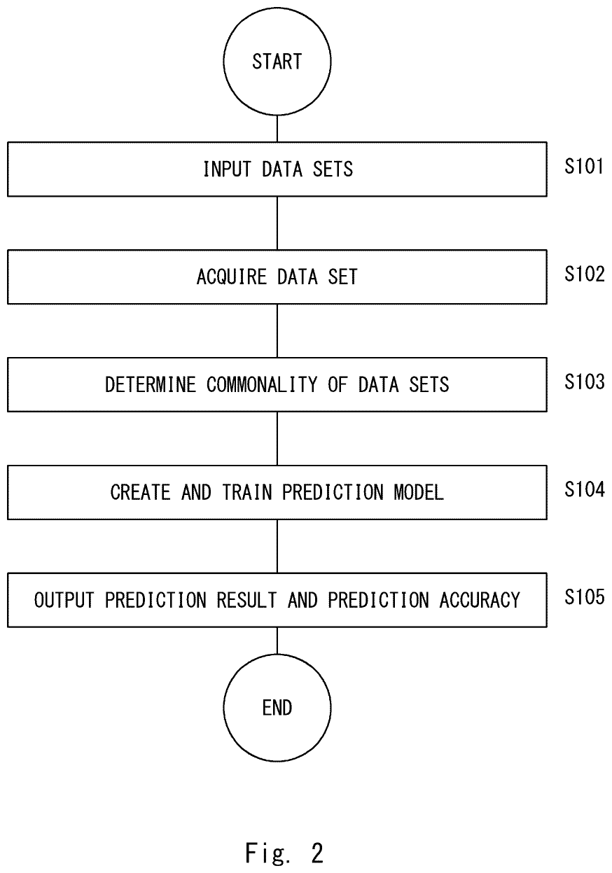

Hereinafter, example embodiments of the present invention will be described with reference to the drawings. FIG. 1 shows a machine learning device according to the present example embodiment, and FIG. 2 shows processing performed by the machine learning device according to the present example embodiment.

A machine learning device 10 shown in FIG. 1 predicts catalyst performance, and includes an input unit 101, an acquisition unit 102, a determination unit 103, a learning unit 104, and an output unit 105.

First, a target data set (target data) including information on an intended catalytic reaction and a plurality of data sets (source data) including information on a catalytic reaction different from the intended catalytic reaction are input to the input unit 101 (S101). In FIG. 1, the former data set is described as a data set A, and the latter data sets are described as a data set B, a data set C, . . . . In the present example embodiment, the latter data sets are described as two types B and C, but the present invention is not limited thereto, and more types of data sets may be input.

These data sets include at least one of chemical reaction information including information on reactants and products, catalyst information including at least one piece of information on catalyst structure such as catalyst composition, catalyst surface area, particle size, and surface structure, and reaction information including at least one of information on reaction conditions such as temperature, pressure, catalyst amount, and gas supply amount, and a reactor size.

In addition, these data sets may include not only chemical reaction information, catalyst information, and reaction information but also catalyst performance information including at least one piece of information on yield, selectivity, and conversion.

Next, the acquisition unit 102 acquires the data sets A to C input to the input unit 101 (S102), and the determination unit 103 determines commonality of the data sets B and C with respect to the data set A (S103). The determination of commonality of the data sets will be described in detail in example embodiment 2.

Next, the learning unit 104 creates a prediction model for predicting catalyst performance using the data set A and a data set selected from the plurality of data sets based on the determination result of S103, and trains the prediction model (S104). At this time, transfer learning is performed with the data set A as target data and the selected data set as source data.

Thereafter, prediction results of the catalyst performance such as a yield, selectivity, and conversion rate and the prediction accuracy of the prediction model are output through the output unit 105 (S105).

As a result, the source data that is the catalytic reaction having high commonality can be used for transfer learning with respect to the target data that is the intended catalytic reaction, and thus machine learning and deep learning can be effectively performed. Therefore, the machine learning device according to the present example embodiment can predict a high performance catalyst. Details of each component and each process will be described in the following example embodiment.

### Example Embodiment 2

In the present example embodiment, a case where chemical reaction information is used for determination of commonality in the determination unit 103 of the machine learning device 10 according to the example embodiment 1 will be described.

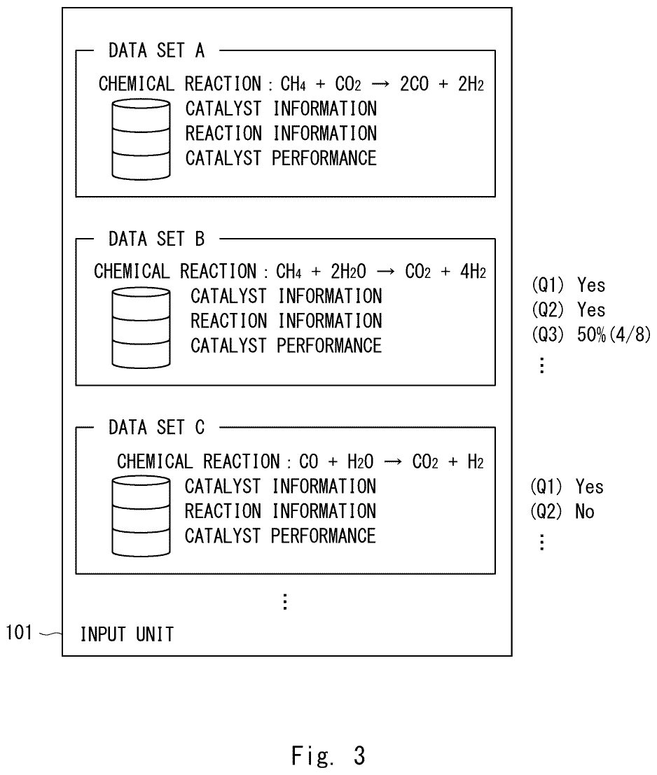

FIG. 3 is a diagram illustrating a data set input to the input unit 101. As information on an intended catalytic reaction, the reaction of formula (1) shown in FIG. 3 is used. In addition, the reactions of formulas (2) and (3) shown in FIG. 3 are used as information on catalytic reactions different from the intended catalytic reaction. Formula (1) as the data set A, formula (2) as the data set B, and formula (3) as the data set C are input to the input unit 101 and transmitted to the acquisition unit 102. Not only these pieces of chemical reaction information but also catalyst information and reaction information may be input as the data sets A to C.

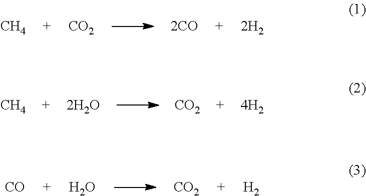

Here, the following questions (Q1) to (Q6) are set as an example of a rule of determination of commonality using chemical reaction information. In terms of priority, the answer to question (Q1) being Yes has the highest priority, followed by the answer to question (Q2) being Yes and the larger answer to (Q3). If the answers to questions (Q2) and (Q3) are the same, the larger answer to (Q4) will rank higher, if the answers to questions (Q2) to (Q4) are the same, the larger answer to (Q5) will rank higher, and if the answers to questions (Q2) to (Q5) are the same, the answer to (Q6) being Yes will rank higher.
(Q1) Is there at least one common compound in reactants and products? (Y/N)
(Q2) Is there a common compound in both reactants and products? (Y/N)
(Q3) What percentage of reactants and products have a common compound? (0 to 100%)
(Q4) What is the maximum number of bonds of a common compound?
(Q5) What is the maximum molecular weight of a common compound?
(Q6) Is a common compound polar molecules? (Y/N)

When the formulas (1) and (2) are compared with each other, the answers to question (Q1) and question (Q2) are Yes, and the answer to question (Q3) is 50% because the formulas (1) and (2) include CH₄ and H₂. Since the number of bonds of CH₄ is four and the number of bonds of H₂ is two, the answer to question (Q4) is four. Since the molecular weight of CH₄ is 16 and the molecular weight of H₂ is 2, the answer to question (Q5) is 16. Since both CH₄ and H₂ are non-polar, the answer to question (Q6) is No.

Similarly, when the formulas (1) and (3) are compared with each other, the answer to question (Q1) is Yes, but the answer to question (Q2) is No because the formulas (1) and (3) include H₂.

Therefore, it is determined that commonality with respect to data set A including formula (1) is higher in data set B including formula (2) than in data set C including formula (3). Then, the data sets are ranked in descending order of commonality, and are transmitted to the learning unit 104 in order from the highest data set.

In the above description, questions (Q1) to (Q6) are cited as the rule of determination of commonality using chemical reaction information, but the determination rule is not limited thereto, and the determination rule may be any rule as long as the rule relates to data input to the input unit 101. For example, the determination rule may relate to catalyst information, reaction information, and the like in addition to chemical reaction information.

The learning unit 104 acquires catalyst information such as catalyst composition and reaction information such as reaction conditions and a reactor size from the transmitted data sets, creates a model for predicting catalyst performance such as a yield, selectivity, and conversion rate, and performs transfer learning.

In addition, the input chemical reaction information may be obtained by sequencing chemical reaction formulas by Reaction SMILES (Simplified Molecular Input Line Entry System) notation, Reaction SMARTS (SMiles ARbitrary Target Specification) notation, or the like. For example, when expressed by Reaction SMILES, Formulas (1) to (3) are represented by the following Formulas (4) to (6), respectively.

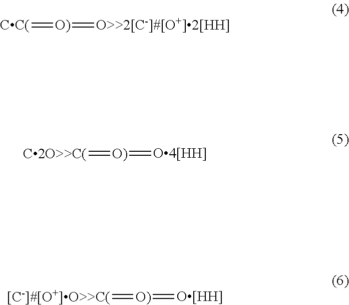

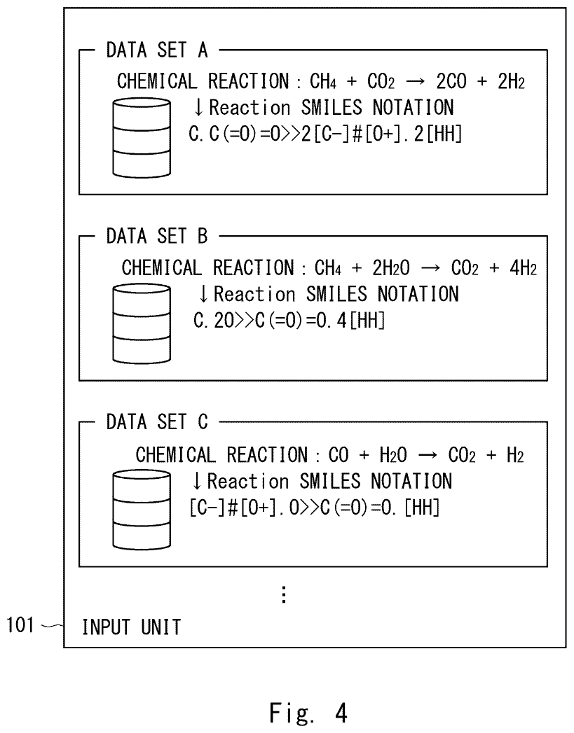

Determination of commonality using sequenced chemical reaction information will be described with reference to FIG. 4. Formulas (4) to (6) obtained by sequencing Formulas (1) to (3) by Reaction SMILES notation are input to the input unit 101 shown in FIG. 4 as chemical reaction information to data sets A to C, respectively.

The determination unit 103 calculates a similarity between a feature amount of the data set A and feature amounts of the data sets B and C using a machine learning model that has learned a feature amount space from these features. Thereafter, the determination unit 103 ranks the data sets based on the similarity, and transmits the data sets to the learning unit 104 in order from the highest data set.

In this way, by ranking the data sets based on the similarity using the sequenced chemical reaction information, it is possible to efficiently extract source data having high commonality, and thus transfer learning is easily performed. Therefore, the machine learning device 10 according to the present example embodiment can predict a high performance catalyst.

Data used when the determination unit 103 calculates a similarity using the machine learning model is not limited to chemical reaction information. For example, the determination unit 103 may calculate a similarity using a machine learning model having at least one of chemical reaction information, catalyst information, and reaction information or a combination thereof as an input. That is, the determination unit 103 may use a machine learning model having any data input to the input unit 101 as an input.

### Example Embodiment 3

In the present example embodiment, a case where catalyst information is used for determination of commonality in the determination unit 103 of the machine learning device 10 according to example embodiments 1 and 2 will be described. Description of components similar to those in example embodiments 1 and 2 may be omitted.

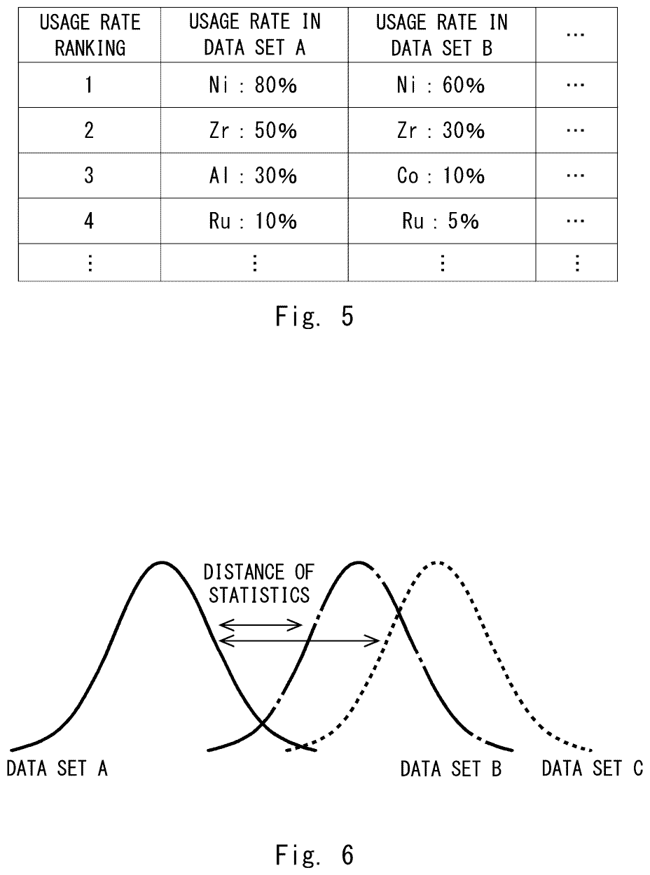

FIG. 5 shows an example in which a usage rate of a catalyst is calculated for each element in each data set and represented in a table. For simplicity, descriptions of data sets C and subsequent data sets are omitted.

The determination unit 103 calculates whether top N elements of the catalysts used in the data set A are common to the data set B and the subsequent data sets. N is any integer and may be determined by a user. In the example shown in FIG. 5, when N=4, the number of common elements in the data set B is three. Thereafter, the determination unit 103 ranks the data sets in descending order of the number of common elements, and sequentially transmits the data sets to the learning unit 104 in order from the highest data set.

Here, as an example of a rule for determining commonality, rules (R1) to (R6) are set in the following priority order.
(R1) A data set having many elements common to top N elements of the data set A is prioritized.
(R2) In a case where data sets have the same number of common elements, a data set that is the same as the element ranked first in the use ranking of the data set A is prioritized.
(R3) In a case where data sets have the same elements in the first use ranking, a data set having a higher usage rate in the first use ranking is prioritized.
(R4) In a case where data sets have the same usage rate, a data set that is the same as the element in the second use ranking of the data set A is prioritized.
(R5) In a case where data sets have the same element in the second use ranking, a data set having a higher usage rate in the second use ranking is prioritized.
(R6) Hereinafter, ranking is similarly performed up to the use ranking N.

In this way, by ranking data sets using catalyst information, it is possible to efficiently extract source data having high commonality, and thus transfer learning is easily performed. The machine learning device according to the present example embodiment can predict a high performance catalyst by using commonality of catalyst information.

### Example Embodiment 4

In the present example embodiment, a case where statistics and distances thereof are used for determination of commonality in the determination unit 103 of the machine learning device 10 according to example embodiments 1 to 3 will be described. Description of components similar to those in example embodiments 1 to 3 may be omitted.

FIG. 6 shows an example representing statistics of each data set. The determination unit 103 calculates statistics (distribution) using any one or a combination of chemical reaction information, catalyst information, and reaction information included in each data set. Thereafter, distances between statistics of the data set A that is target data and statistics of the data set B and subsequent data sets that are source data are calculated, and the data sets are ranked in ascending order of the distance.

In FIG. 6, the statistics of the data set A are indicated by a solid line, the statistics of the data set B are indicated by a broken line, the statistics of the data set C are indicated by a dotted line, and the distances to the data set A are calculated from the data of these statistics. The statistics and the distances between the statistics are calculated by using a measure for measuring a distance between distributions such as Kalback library divergence.

Since the shorter the distance to the statistics of the data set A, the higher commonality of the data, the data sets can be ranked based on the distances of the statistics, source data having high commonality can be efficiently extracted, and transfer learning can be easily performed. Therefore, the machine learning device according to the present example embodiment can predict a high performance catalyst.

### Example Embodiment 5

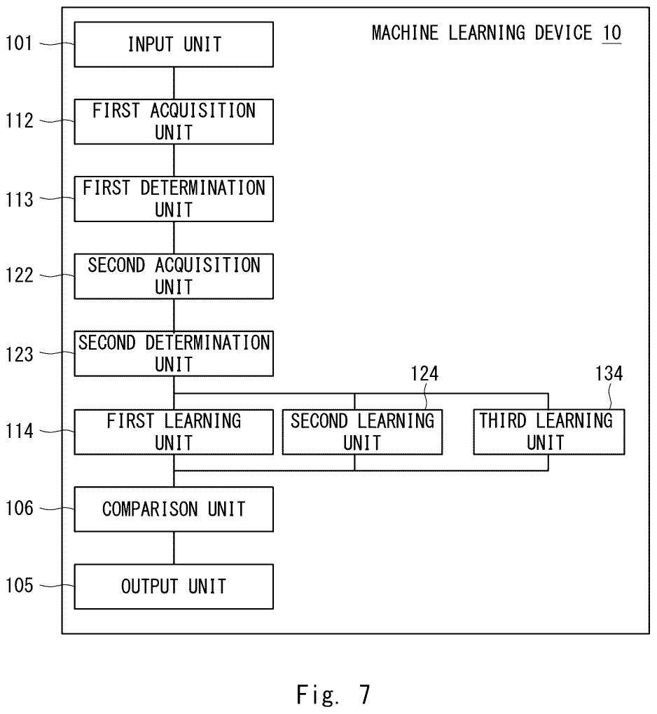

In the present example embodiment, the machine learning device 10 according to example embodiments 1 to 4 will be described in more detail. FIG. 7 illustrates the machine learning device according to the present example embodiment, and the machine learning device 10 includes an input unit 101, a first acquisition unit 112, a first determination unit 113, a second acquisition unit 122, a second determination unit 123, a first learning unit 114, a second learning unit 124, a third learning unit 134, an output unit 105, and a comparison unit 106.

The input unit 101 and the output unit 105 are the same components as those in the example embodiments 1 to 4, and the first acquisition unit 112 and the first learning unit 114 have functions equivalent to those of the acquisition unit 102 and the learning unit 104 in the example embodiment 1, and thus detailed description thereof will be omitted.

The first determination unit 113 transmits only a data set including a common compound among the data set B and subsequent data sets which are source data transmitted from the first acquisition unit 112 to the second acquisition unit 122. That is, a data set for which the answer to question (Q1) is No in the example embodiment 2 is excluded at this stage. This makes it possible to efficiently select source data.

Exclusion of data sets by the first determination unit 113 can obtain a large effect, for example, in a case where a large amount of source data stored in a server or the like is extracted via the input unit 101.

For the data set transmitted to the second acquisition unit 122, the second determination unit 123 executes questions (Q2) and subsequent questions in the example embodiment 2, and transmits obtained information to the first learning unit 114, the second learning unit 124, and the third learning unit 134.

Here, the first learning unit 114 creates a prediction model by processing similar to that of the learning unit 104 in the example embodiments 1 to 4. The second learning unit 124 regards the data set A that is the target data and the data set B that is the source data as the same system and combines the data set A and the data set B to create a prediction model. The third learning unit 134 creates a prediction model using only the data set A. That is, the first learning unit 114 creates a prediction model subjected to transfer learning, the second learning unit 124 creates a prediction model based on combined data, and the third learning unit 134 creates a prediction model for only target data.

Each prediction model is transmitted to the comparison unit 106, and the comparison unit 106 compares the models. As a result, it is possible to obtain information indicating which of the transfer learning, the combined data, and the target data has the highest prediction accuracy. When the combined data has the highest accuracy, it is also possible to loop the machine learning method of the present disclosure using the combined data as new target data.

In addition, since optimal source data can be selected from the actual prediction results, the prediction accuracy at this time can also be achieved. It is possible to feed back the determination criteria of the second determination unit 123, that is, the questions (Q2) to (Q6) in the example embodiment 2 based on such prediction results.

In addition, by performing Bayesian optimization together, even in a case where there are few known data, it is possible to predict information of a catalyst composition having high catalyst performance and reaction information such as reaction conditions and a reactor size, which can lead to acceleration of catalyst development.

In addition, by performing Bayesian optimization together, even in a case where there are few known data, it is possible to predict information of a catalyst composition having high catalyst performance and reaction information such as reaction conditions and a reactor size, which can lead to acceleration of catalyst development.

In the above-described example, a program includes a group of instructions (or software code) for causing a computer to execute one or more functions described in the example embodiments when being read by the computer. The program may be stored in a non-transitory computer-readable medium or in a tangible storage medium. By way of example, and not limitation, computer-readable media or tangible storage media include a random-access memory (RAM), a read-only memory (ROM), a flash memory, a solid-state drive (SSD) or other memory technology, a CD-ROM, a digital versatile disc (DVD), a Blu-ray (registered trademark) disc or other optical disk storage, a magnetic cassette, a magnetic tape, a magnetic disk storage, or other magnetic storage devices. The program may be transmitted on a transitory computer readable medium or a communication medium. By way of example, and not limitation, transitory computer-readable or communication media include electrical, optical, acoustic, or other forms of propagated signals.

While the present disclosure has been particularly shown and described with reference to example embodiments thereof, the present disclosure is not limited to these example embodiments. It will be understood by those of ordinary skill in the art that various changes in form and details may be made therein without departing from the spirit and scope of the present disclosure as defined by the claims. And each embodiment can be appropriately combined with other embodiments.

Each drawing is merely illustrative for describing one or more example embodiments. Each drawing is not associated with only one particular example embodiment, but may be associated with one or more other example embodiments. As one of ordinary skill in the art will appreciate, various features or steps described with reference to any one of the figures may be combined with features or steps shown in one or more other figures, for example, to create an example embodiment not explicitly shown or described. All of the features or steps shown in any one of the figures for describing exemplary example embodiments are not necessarily mandatory, and some features or steps may be omitted. The order of the steps described in any of the figures may be changed as appropriate.

Some or all of the above-described example embodiments may be described as the following supplementary notes, but are not limited to the following supplementary notes.

### Supplementary Note 1

A machine learning device including:
an acquisition unit configured to acquire a target data set including information on an intended catalytic reaction and a plurality of data sets including information on catalytic reactions different from the intended catalytic reaction;
a determination unit configured to determine commonality regarding catalytic reactions between the target data set and each of the plurality of data sets; and
a learning unit configured to train a prediction model for predicting catalyst performance from the plurality of data sets by using a data set selected based on the determination result and the target data set.

### Supplementary Note 2

The machine learning device according to Supplementary Note 1, wherein the target data set and the plurality of data sets include at least one of chemical reaction information including information on reactants and products, catalyst information including at least one of information on a catalyst composition and information on a catalyst structure, and reaction information including at least one of information on reaction conditions and information on a reactor size, and the determination unit determines commonality by using at least one of the chemical reaction information, the catalyst information, and the reaction information.

### Supplementary Note 3

The machine learning device according to Supplementary Note 2, wherein the determination unit ranks the plurality of data sets based on a rule using at least one of the chemical reaction information, the catalyst information, and the reaction information.

### Supplementary Note 4

The machine learning device according to Supplementary Note 2, wherein the determination unit calculates a similarity between a feature amount of the target data set and a feature amount of each of the plurality of data sets by using a machine learning model that has learned a feature amount space indicating at least one feature of the chemical reaction information, the catalyst information, and the reaction information, and ranks the plurality of data sets based on the similarity.

### Supplementary Note 5

The machine learning device according to Supplementary Note 2, wherein the determination unit calculates a distance between statistics of the target data set calculated based on at least one of the chemical reaction information, the catalyst information, and the reaction information and statistics of each of the plurality of data sets, and ranks the plurality of data sets based on the distance.

### Supplementary Note 6

A machine learning method including:
acquiring a target data set including information on an intended catalytic reaction and a plurality of data sets including information on catalytic reactions different from the intended catalytic reaction;
determining commonality regarding catalytic reactions between the target data set and each of the plurality of data sets; and
training a prediction model for predicting catalyst performance from the plurality of data sets by using a data set selected based on the determination result and the target data set.

### Supplementary Note 7

The machine learning method according to Supplementary Note 6, wherein the target data set and the plurality of data sets include at least one of chemical reaction information including information on reactants and products, catalyst information including at least one of information on a catalyst composition and information on a catalyst structure, and reaction information including at least one of information on reaction conditions and information on a reactor size, and commonality is determined by using at least one of the chemical reaction information, the catalyst information, and the reaction information.

### Supplementary Note 8

The machine learning method according to Supplementary Note 7, wherein the plurality of data sets are ranked based on a rule using at least one of the chemical reaction information, the catalyst information, and the reaction information.

### Supplementary Note 9

The machine learning method according to Supplementary Note 7, wherein
a similarity between a feature amount of the target data set and a feature amount of each of the plurality of data sets is calculated by using a machine learning model that has learned a feature amount space indicating at least one feature of the chemical reaction information, the catalyst information, and the reaction information, and
the plurality of data sets are ranked based on the similarity.

### Supplementary Note 10

The machine learning method according to Supplementary Note 7, wherein a distance between statistics of the target data set calculated based on at least one of the chemical reaction information, the catalyst information, and the reaction information and statistics of each of the plurality of data sets is calculated, and the plurality of data sets are ranked based on the distance.

### Supplementary Note 11

A program causing a computer included in a machine learning device to execute:
a step of acquiring a target data set including information on an intended catalytic reaction and a plurality of data sets including information on catalytic reactions different from the intended catalytic reaction;
a step of determining commonality regarding catalytic reactions between the target data set and each of the plurality of data sets; and
a step of training a prediction model for predicting catalyst performance from the plurality of data sets by using a data set selected based on the determination result and the target data set.

### Supplementary Note 12

The program according to Supplementary Note 11, wherein the target data set and the plurality of data sets include at least one of chemical reaction information including information on reactants and products, catalyst information including at least one of information on a catalyst composition and information on a catalyst structure, and reaction information including at least one of information on reaction conditions and information on a reactor size, and the computer is caused to execute a step of determining commonality by using at least one of the chemical reaction information, the catalyst information, and the reaction information.

### Supplementary Note 13

The program according to Supplementary Note 12, wherein the computer is caused to execute a step of ranking the plurality of data sets based on a rule using at least one of the chemical reaction information, the catalyst information, and the reaction information.

### Supplementary Note 14

The program according to Supplementary Note 12, wherein the computer is caused to execute a step of calculating a similarity between a feature amount of the target data set and a feature amount of each of the plurality of data sets by using a machine learning model that has learned a feature amount space indicating at least one feature of the chemical reaction information, the catalyst information, and the reaction information, and ranking the plurality of data sets based on the similarity.

### Supplementary Note 15

The program according to Supplementary Note 12, wherein the computer is caused to execute a step of calculating a distance between statistics of the target data set calculated based on at least one of the chemical reaction information, the catalyst information, and the reaction information and statistics of each of the plurality of data sets, and ranking the plurality of data sets based on the distance.

### Supplementary Note 16

A machine learning system including:
a server; and
a machine learning device,
wherein the machine learning device includes:
an acquisition unit configured to acquire a target data set including information on an intended catalytic reaction and a plurality of data sets including information on catalytic reactions different from the intended catalytic reaction from the server;
a determination unit configured to determine commonality regarding catalytic reactions between the target data set and each of the plurality of data sets; and
a learning unit configured to train a prediction model for predicting catalyst performance from the plurality of data sets by using a data set selected based on the determination result and the target data set.

### Supplementary Note 17

The machine learning system according to Supplementary Note 16, wherein the target data set and the plurality of data sets include at least one of chemical reaction information including information on reactants and products, catalyst information including at least one of information on a catalyst composition and information on a catalyst structure, and reaction information including at least one of information on reaction conditions and information on a reactor size, and the determination unit determines commonality by using at least one of the chemical reaction information, the catalyst information, and the reaction information.

### Supplementary Note 18

The machine learning system according to Supplementary Note 17, wherein the determination unit ranks the plurality of data sets based on a rule using at least one of the chemical reaction information, the catalyst information, and the reaction information.

### Supplementary Note 19

The machine learning system according to Supplementary Note 17, wherein the determination unit calculates a similarity between a feature amount of the target data set and a feature amount of each of the plurality of data sets by using a machine learning model that has learned a feature amount space indicating at least one feature of the chemical reaction information, the catalyst information, and the reaction information, and ranks the plurality of data sets based on the similarity.

### Supplementary Note 20

The machine learning system according to Supplementary Note 17, wherein the determination unit calculates a distance between statistics of the target data set calculated based on at least one of the chemical reaction information, the catalyst information, and the reaction information and statistics of each of the plurality of data sets, and ranks the plurality of data sets based on the distance.

Some or all of the elements (for example, configurations and functions) described in the Supplementary Notes 2 to 5 dependent on the machine learning device described in the Supplementary Note 1 can also be dependent on the machine learning method described in the Supplementary Notes 6 to 10, the program described in the Supplementary Notes 11 to 15, and the machine learning system described in the Supplementary Notes 16 to 20 by a similar dependency. Some or all of the elements described in any supplementary note may be applied to various hardware, software, recording means for recording software, systems, and methods.

This application is based upon and claims the benefit of priority from Japanese patent application No. 2023-018739, filed on Feb. 9, 2023, the disclosure of which is incorporated herein in its entirety by reference.

### REFERENCE SIGNS LIST

10 machine learning device
10 input unit
102 acquisition unit
103 determination unit
104 learning unit
105 output unit
106 comparison unit
112 first acquisition unit
113 first determination unit
114 first learning unit
122 second acquisition unit
123 second determination unit
124 second learning unit
134 third learning unit

1. A machine learning device comprising:
a memory configured to store instructions; and
a processor configured to execute the instructions to:
acquire a target data set including information on an intended catalytic reaction and a plurality of data sets including information on catalytic reactions different from the intended catalytic reaction;
determine commonality regarding catalytic reactions between the target data set and each of the plurality of data sets; and
train a prediction model for predicting catalyst performance from the plurality of data sets by using a data set selected based on the determination result and the target data set.

2. The machine learning device according to claim 1, wherein the target data set and the plurality of data sets include at least one of chemical reaction information including information on reactants and products, catalyst information including at least one of information on a catalyst composition and information on a catalyst structure, and reaction information including at least one of information on reaction conditions and information on a reactor size, and the processor is further configured to execute the instructions to determine commonality by using at least one of the chemical reaction information, the catalyst information, and the reaction information.

3. The machine learning device according to claim 2, wherein the processor is further configured to execute the instructions to rank the plurality of data sets based on a rule using at least one of the chemical reaction information, the catalyst information, and the reaction information.

4. The machine learning device according to claim 2, wherein the processor is further configured to execute the instructions to calculate a similarity between a feature amount of the target data set and a feature amount of each of the plurality of data sets by using a machine learning model that has learned a feature amount space indicating at least one feature of the chemical reaction information, the catalyst information, and the reaction information, and ranks the plurality of data sets based on the similarity.

5. The machine learning device according to claim 2, wherein the processor is further configured to execute the instructions to calculate a distance between statistics of the target data set calculated based on at least one of the chemical reaction information, the catalyst information, and the reaction information and statistics of each of the plurality of data sets, and ranks the plurality of data sets based on the distance.

6. A machine learning method comprising:
acquiring a target data set including information on an intended catalytic reaction and a plurality of data sets including information on catalytic reactions different from the intended catalytic reaction;
determining commonality regarding catalytic reactions between the target data set and each of the plurality of data sets; and
training a prediction model for predicting catalyst performance from the plurality of data sets by using a data set selected based on the determination result and the target data set.

7. The machine learning method according to claim 6, wherein the target data set and the plurality of data sets include at least one of chemical reaction information including information on reactants and products, catalyst information including at least one of information on a catalyst composition and information on a catalyst structure, and reaction information including at least one of information on reaction conditions and information on a reactor size, and commonality is determined by using at least one of the chemical reaction information, the catalyst information, and the reaction information.

8. The machine learning method according to claim 7, wherein the plurality of data sets are ranked based on a rule using at least one of the chemical reaction information, the catalyst information, and the reaction information.

9. The machine learning method according to claim 7, wherein
a similarity between a feature amount of the target data set and a feature amount of each of the plurality of data sets is calculated by using a machine learning model that has learned a feature amount space indicating at least one feature of the chemical reaction information, the catalyst information, and the reaction information, and
the plurality of data sets are ranked based on the similarity.

10. The machine learning method according to claim 7, wherein a distance between statistics of the target data set calculated based on at least one of the chemical reaction information, the catalyst information, and the reaction information and statistics of each of the plurality of data sets is calculated, and the plurality of data sets are ranked based on the distance.

11. A non-transitory computer-readable medium storing a program causing a computer included in a machine learning device to execute:
a step of acquiring a target data set including information on an intended catalytic reaction and a plurality of data sets including information on catalytic reactions different from the intended catalytic reaction;
a step of determining commonality regarding catalytic reactions between the target data set and each of the plurality of data sets; and
a step of training a prediction model for predicting catalyst performance from the plurality of data sets by using a data set selected based on the determination result and the target data set.

12. The non-transitory computer-readable medium according to claim 11, wherein the target data set and the plurality of data sets include at least one of chemical reaction information including information on reactants and products, catalyst information including at least one of information on a catalyst composition and information on a catalyst structure, and reaction information including at least one of information on reaction conditions and information on a reactor size, and the computer is caused to execute a step of determining commonality by using at least one of the chemical reaction information, the catalyst information, and the reaction information.

13. The non-transitory computer-readable medium according to claim 12, wherein the computer is caused to execute a step of ranking the plurality of data sets based on a rule using at least one of the chemical reaction information, the catalyst information, and the reaction information.

14. The non-transitory computer-readable medium according to claim 12, wherein the computer is caused to execute a step of calculating a similarity between a feature amount of the target data set and a feature amount of each of the plurality of data sets by using a machine learning model that has learned a feature amount space indicating at least one feature of the chemical reaction information, the catalyst information, and the reaction information, and ranking the plurality of data sets based on the similarity.

15. The non-transitory computer-readable medium according to claim 12, wherein the computer is caused to execute a step of calculating a distance between statistics of the target data set calculated based on at least one of the chemical reaction information, the catalyst information, and the reaction information and statistics of each of the plurality of data sets, and ranking the plurality of data sets based on the distance.

16-20. (canceled)
